(19) United States
(12) Patent Application Publication
(10) Pub. No.: US 2023/0240602 A1
(43) Pub. Date: Aug. 3, 2023

(54) INDUCING HYPOXIA TO MONITOR ONE OR MORE PATIENT CONDITIONS USING A MEDICAL DEVICE SYSTEM

(71) Applicants: Covidien LP, Mansfield, MA (US); Hemex Health, Inc., Portland, OR (US)

(72) Inventors: Derek L. Moody, Lafayette, CO (US); Jacob D. Dove, Lafayette, CO (US); Paul S. Addison, Edinburgh (GB); Rakesh K. Sethi, Vancouver (CA); Peter M. Galen, Portland, OR (US)

(21) Appl. No.: 18/151,919

(22) Filed: Jan. 9, 2023

Related U.S. Application Data
(60) Provisional application No. 63/301,828, filed on Jan. 21, 2022.

(51) Int. Cl. A61B 5/00; A61B 5/0205

(52) U.S. Cl. CPC A61B 5/4884; A61B 5/0059; A61B 5/0205; A61B 5/6826; A61B 5/02255; A61B 2562/0238

(57) ABSTRACT

This disclosure is directed to devices, systems, and techniques for monitoring one or more patient conditions. For example, a system includes a memory and processing circuitry communicatively coupled to the memory. The processing circuitry is configured to receive, from a sensor, an electrical representation of a first optical signal and control a pressure device to apply pressure to the patient in order to affect one or more physiological parameters proximate to the sensor. Additionally, the processing circuitry is configured to receive, from the sensor after applying the pressure to the patient, an electrical representation of a second optical signal; and determine, based on the electrical representation of the first optical signal and the electrical representation of the second optical signal, one or more patient conditions.

This application claims the benefit of U.S. Provisional Pat. Application No. 63/301,828, filed on Jan. 21, 2022, the entire content of which is incorporated herein by reference.

### TECHNICAL FIELD

The disclosure relates generally to medical device systems and, more particularly, medical device systems configured to monitor patient parameters.

### BACKGROUND

Some types of medical devices may be used to monitor one or more physiological parameters of a patient. Such medical devices may include, or may be part of a system that includes, sensors that detect signals associated with such physiological parameters. Values determined based on such signals may be used to assist in detecting changes in patient conditions, in evaluating the efficacy of a therapy, or in generally evaluating patient health.

### SUMMARY

In general, the disclosure is directed to devices, systems, and techniques for monitoring one or more patient conditions using a medical device system. For example, the medical device system may include an optical sensor configured to perform one or more optical signal measurements. Optical signals captured by the optical sensor may indicate one or more physiological parameters such as heart rate, respiration rate, pulse oximetry (SpO₂), regional oxygen saturation (rSO₂), pulse transit time (PTT), a photoplethysmography (PPG) value, blood pressure, hemoglobin content value, or any combination thereof. Techniques described herein may include determining these one or more physiological parameters in order to one or more patient conditions such as sickle cell anemia, aplastic anemia, iron deficiency anemia, thalassemia, vitamin deficiency anemia, or any combination thereof. In some examples, the medical device system may include one or more wearable devices, but this is not required. Techniques described herein may be performed by one or more wearable devices and/or one or more devices that are not wearable.

Some patient conditions may affect the patient’s blood cells. For example, anemia may decrease a concentration of hemoglobin in the bloodstream, thus decreasing a rate that the blood can deliver oxygen to the brain as compared with people that have a normal concentration of hemoglobin in the bloodstream. Consequently, to monitor anemia and similar conditions, it may be beneficial to monitor one or more physiological parameters that indicate a concentration of hemoglobin and/or indicate one or more other biometric characteristics of the patient. Optical sensors may be configured to generate data that indicates the one or more physiological parameters, thus allowing the system to monitor patient conditions. Monitoring one or more patient conditions may, in some examples, comprise performing a single signal measurement or a few signal measurements in order to perform a “spot check” to obtain a current status of the one or more patient conditions. In some examples, monitoring one or more patient conditions may include performing a set of signal measurements over a period of time in order to determine a trend in the patient condition over the period of time.

A medical device system may be configured to perform one or more optical signal measurements using an optical sensor to collect optical data. The optical sensor may include one or more light emitters and one or more light detectors. To perform an optical signal measurement using the optical sensor, the one or more light emitters may emit an output optical signal comprising a plurality of output optical wavelength signals to the area of the patient’s body. The one or more light detectors may receive a return optical signal comprising a plurality of return optical wavelength signals. Based on the output optical signal and the return optical signal, processing circuitry may determine the one or more physiological parameters of the patient. The optical sensor may be included as part of the medical device system such that a wearable device may secure the optical sensor to a part of the patient’s body. The medical device system does not need to include a wearable device. In some examples, the medical device system may include an optical sensor without including a medical device. The optical sensor may emit output optical signals into the patient’s body and receive the return optical signals to the patient’s body.

The techniques of this disclosure may provide one or more advantages. In some examples, the medical device system may include a pressure device that automatically applies pressure to the patient in order to perform one or more optical signal measurements. For example, it may be beneficial to induce hypoxia in an area of the patient where the optical sensor(s) perform optical signal measurements. Inducing hypoxia may increase an ability of the system to track patient conditions based on the measured optical signals as compared with systems that do not induce hypoxia. Techniques described herein may include performing measurements after inducing hypoxia, performing measurements without inducing hypoxia, performing measurements after releasing hypoxia-inducing pressure, or any combination thereof. The techniques of this disclosure provide for non-invasive and substantially real time analysis of one or more patient states based on data corresponding to the one or more optical signal measurements. Automatically monitoring data corresponding to one or more optical signal measurements may be more effective for tracking one or more conditions (e.g., anemia) in real time as compared with one or more other techniques such as blood tests and laboratory analysis.

In some examples, a system includes a memory and processing circuitry communicatively coupled to the memory. The processing circuitry is configured to receive, from a sensor, an electrical representation of a first optical signal and control a pressure device to apply pressure to the patient in order to affect one or more physiological parameters proximate to the sensor. Additionally, the processing circuitry is configured to receive, from the sensor after applying the pressure to the patient, an electrical representation of a second optical signal; and determine, based on the electrical representation of the first optical signal and the electrical representation of the second optical signal, one or more patient conditions.

In some examples, a method includes receiving, by processing circuitry communicatively coupled to a memory, an electrical representation of a first optical signal and controlling, by the processing circuitry, a pressure device to apply pressure to the patient in order to affect one or more physiological parameters proximate to the sensor. Additionally, the method comprises receiving, by the processing circuitry from the sensor after applying the pressure to the patient, an electrical representation of a second optical signal; and determining, by the processing circuitry based on the electrical representation of the first optical signal and the electrical representation of the second optical signal, one or more patient conditions.

In some examples, a non-transitory computer-readable medium comprising instructions for causing one or more processors to: receive an electrical representation of a first optical signal; control a pressure device to apply pressure to the patient in order to affect one or more physiological parameters proximate to the sensor; receive, from the sensor after applying the pressure to the patient, an electrical representation of a second optical signal; and determine, based on the electrical representation of the first optical signal and the electrical representation of the second optical signal, one or more patient conditions.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF DRAWINGS

FIG. 1 illustrates the environment of an example medical device system in conjunction with a patient, in accordance with one or more techniques of this disclosure.

FIG. 2 is a block diagram illustrating an example configuration of components of an external device system, in accordance with one or more techniques of this disclosure.

FIG. 3 is a block diagram illustrating an example configuration of components of a medical device system, in accordance with one or more techniques of this disclosure.

FIG. 4 is a conceptual drawing illustrating an example configuration of a wearable device of the medical device system of FIG. 1 and FIG. 3, in accordance with one or more techniques of this disclosure.

FIG. 5 is a conceptual drawing illustrating an example location of pressure device 52 and wearable device 40 on an arm 6 of patient 4, in accordance with one or more techniques of this disclosure.

FIG. 6 is a block diagram illustrating an example system that includes an access point, a network, external computing devices, such as a server, and one or more other computing devices, which may be coupled to an external device system, processing circuitry, and a medical device system via network, in accordance with one or more techniques of this disclosure.

FIG. 7 is a flow diagram illustrating a first example operation for monitoring a patient condition, in accordance with one or more techniques of this disclosure.

FIG. 8 is a flow diagram illustrating a second example operation for monitoring a patient condition, in accordance with one or more techniques of this disclosure.

Like reference characters denote like elements throughout the description and figures.

### DETAILED DESCRIPTION

This disclosure describes techniques for performing one or more optical signal measurements using a medical device system that includes one or more optical sensors and one or more pressure devices. Processing circuitry may control the optical sensor(s) and the pressure device(s) in order to cause the optical sensor(s) to collect optical signals. The processing circuitry may receive one or more electrical representations of these optical signals, and track the electrical representations of the optical signals in order to monitor one or more patient conditions. To control the optical sensor(s) to perform one or more optical signal measurements, the processing circuitry may control the pressure device(s) in order to apply pressure to a patient in order to induce hypoxia in an area proximate to an optical sensor. The system may perform measurements after inducing hypoxia, perform measurements without inducing hypoxia, perform measurements after releasing hypoxia-inducing pressure, or any combination thereof.

It may be beneficial to monitor anemia, or other hemoglobin-related conditions such as sickle cell anemia, sickle cell trait, and beta thalassemia using a non-invasive medical device system. One or more techniques described herein include improving a sensitivity of the medical device system to one or more patient conditions. For example, locally induced hypoxia affects a concentration of hemoglobin in the bloodstream. A magnitude of the effect of locally induced hypoxia on hemoglobin concentration may indicate a status of one or more patient conditions. Applying and releasing pressure may induce a hypoxic environment. This local hypoxic environment could be beneficial for monitoring hemoglobin-related patient conditions such as measuring a level of hemoglobin for anemia, measuring sickle cell anemia or sickle cell trait, or measuring beta thalassemia. In addition to creating a hypoxic environment, one or more parameters corresponding to a return to normal blood flow after induced hypoxia may indicate a status of one or more patient conditions. One or more measured parameters may indicate arterial stiffness, Pulse Transit Time (PTT), vascular resistance, capillary refill, fluid responsivity, a new vascular measure of the filling and emptying rate, and a viscosity of the blood after a period in which the blood is stagnant during hypoxia.

In some examples, a medical device system may include a combination of an optical sensor configured to collect one or more optical signals having various wavelengths and a pressure device. The system may determine one or more physiological parameters based on the optical signals generated by the optical sensor. For example, processing circuitry of the system may receive an electrical representation of an optical signal collected by the optical signal. As described herein, an “electrical representation of an optical signal” may comprise an electrical signal that indicates one or more parameters of the optical signal such that processing circuitry may analyze the electrical signal in order to determine the one or more parameters of the optical signal.

In some examples, the system may control the pressure device to apply a pressure to the subject’s finger or arm. This pressure may, in some examples, depend on a blood pressure of the patient. This blood pressure may be tracked at a variety of sites of the patient such as the arm or finger as part of the feedback control. In some examples, feedback control may depend on a plethysmograph signal. One or more parameters derived from electrical representations of measured optical signals may be included as a part of a feedback control to adjust the pressure applied to the patient using the pressure device. In some examples, a feedback control loop for controlling the pressure device to apply pressure may analyze an electrical representation of an optical signal to determine one or more response curves of the optical signal based on a magnitude of the applied pressure. In some examples, the feedback control loop may depend on a rate of change of the optical signal over time or the alteration of a curve morphology over multiple cycles.

The system, in some examples, may obtain an electrical representation of a first optical signal. For example, an optical sensor may collect the first optical signal and generate the electrical representation of the first optical signal for output to processing circuitry of the system. The first optical signal may represent a baseline optical signal that a wearable device may collect when the pressure device is not applying pressure to the patient. The first optical signal is not limited to being collected by a wearable device. One or more sensors that are not part of a wearable device may collect the first optical signal. The system may control the pressure device to apply pressure. The pressure may, in some cases, induce hypoxia in an area of the patient where one or more sensors collect one or more optical signals.

The system may obtain an electrical representation of a second optical signal when the pressure device applies pressure in order to obtain data when the area is under a state of hypoxia. For example, an optical sensor may collect the second optical signal and generate the electrical representation of the second optical signal for output to processing circuitry of the system. The second optical signal may represent an instantaneous reading or a reading over a period of time while hypoxia is induced. The system may determine one or more physiological parameters based on the electrical representation of the first optical signal and/or the electrical representation of the second optical signal. The system may control the pressure device to release pressure from the patient over a period of time. The system may obtain an electrical representation of a third optical signal corresponding to a third measurement. The optical sensor may collect the electrical representation of the third optical signal at a specific point in time or over a time period. In some examples, the electrical representation of the third optical signal may be collected during a period of time that at least partially overlaps with the period of time when the pressure is released. The system may determine one or more physiological parameters based on any one or combination of the electrical representation of the first optical signal, the electrical representation of the second optical signal, and the electrical representation of the third optical signal.

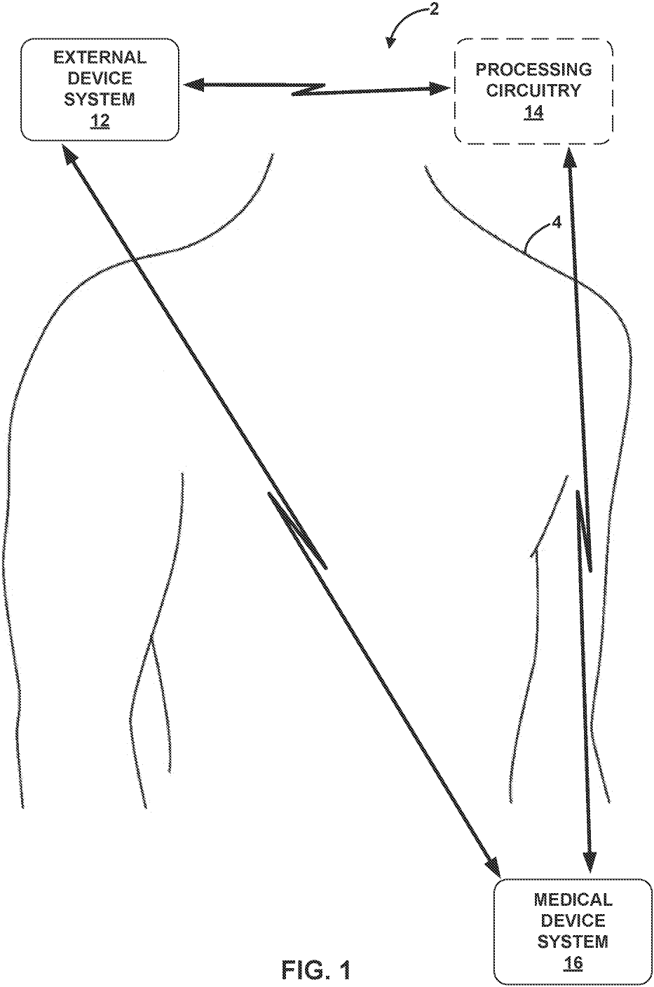

FIG. 1 illustrates the environment of an example medical device system 2 in conjunction with a patient 4, in accordance with one or more techniques of this disclosure. The example techniques may be used with an external device system 12, processing circuitry 14, a medical device system 16, other devices not illustrated in FIG. 1, or any combination thereof. One or both of external device system 12 and medical device system 16 may include at least a portion of processing circuitry 14.

In some examples, the medical device system 2 may collect data that medical device system 2 can analyze in order to monitor one or more patient conditions. In some examples, medical device system 2 may use one or more physiological sensors, such as electrodes, optical sensors, chemical sensors, temperature sensors, acoustic sensors, motion sensors, or any combination thereof. Data collected by the one or more physiological sensors may be indicative of one or more physiological parameters. For example, the one or more physiological parameters may include one or more parameters corresponding to the patient’s cardiovascular health and/or one or more parameters corresponding to the patient’s respiratory health. These parameters may indicate aspects of one or more patient conditions such as sickle cell anemia, aplastic anemia, iron deficiency anemia, thalassemia, vitamin deficiency anemia, or any combination thereof. Medical device system 2 may measure the one or more parameters based on the collected data. Users (e.g., a clinician and/or the patient) may view information corresponding to data measurements using the external device system 12 and users may control one or more aspects of medical device system 2 using the external device system 12. For example, the medical device system 2 may control medical device system 12 to perform one or more measurements based on user input to external device system 12.

External device system 12 may be a computing device with a display viewable by the user and an interface for providing input to external device system 12 (i.e., a user input mechanism). For example, external device system 12 may include a display screen (e.g., a liquid crystal display (LCD) or a light emitting diode (LED) display) that presents information to the user. In addition, external device system 12 may include a touch screen display, keypad, buttons, a peripheral pointing device, voice activated microphone, or another input mechanism that allows the user to navigate through the user interface of external device system 12 and provide input. If external device system 12 includes buttons and a keypad, the buttons may be dedicated to performing a certain function, e.g., a power button, the buttons and the keypad may be soft keys that change in function depending upon the section of the user interface currently viewed by the user, or any combination thereof.

In other examples, external device system 12 may be a larger workstation or a separate application within another multi-function device, rather than a dedicated computing device. For example, the multi-function device may be a notebook computer, tablet computer, workstation, one or more servers, cellular phone, personal digital assistant, or another computing device that may run an application that enables the computing device to operate as a secure device.

When external device 12 is configured for use by a clinician, external device 12 may be used to transmit instructions to medical device system 16. Example instructions may include requests to perform one or more measurements and any other information that may be useful for programming medical device system 16. The clinician may also configure and store operational parameters for medical device system 16 within medical device system 16 with the aid of external device 12. In some examples, external device 12 assists the clinician in the configuration of medical device system 16 by identifying potentially beneficial operational parameter values.

Whether external device 12 is configured for clinician or patient use, external device 12 is configured to communicate with medical device system 16 and, optionally, another computing device (not illustrated in FIG. 1), via wireless communication. External device 12, for example, may communicate via near-field communication technologies (e.g., inductive coupling, near-field communication (NFC) or other communication technologies operable at ranges less than 10-20 cm) and far-field communication technologies (e.g., RF telemetry according to the 802.11 or Bluetooth® specification sets, Wi-Fi, 3G, 4G, LTE, 5G or other communication technologies operable at ranges greater than near-field communication technologies). For example, external device 12 may send data, such as data received from medical device system 16, to another external device such as a smartphone, a tablet, or a desktop computer, and the other external device may in turn send the data to the computer network. In other examples, external device 12 may directly communicate with the computer network without an intermediary device.

Processing circuitry 14, in some examples, may include one or more processors that are configured to implement functionality and/or process instructions for execution within external device system 12, medical device system 16, or other devices not illustrated in FIG. 1. For example, processing circuitry 14 may be capable of processing instructions stored in a storage device. Processing circuitry 14 may include, for example, microprocessors, digital signal processors (DSPs), application specific integrated circuits (ASICs), field-programmable gate arrays (FPGAs), or equivalent discrete or integrated logic circuitry, or a combination of any of the foregoing devices or circuitry. Accordingly, processing circuitry 14 may include any suitable structure, whether in hardware, software, firmware, or any combination thereof, to perform the functions ascribed herein to processing circuitry 14.

Processing circuitry 14 may represent processing circuitry located within any one or combination of external device system 12, medical device system 16, or other devices not illustrated in FIG. 1. In some examples, processing circuitry 14 may be entirely located within external device system 12. In other examples, processing circuitry 14 may be entirely located within medical device system 16. In other examples, processing circuitry 14 may be entirely located within one or more other systems not illustrated in FIG. 1. In other examples, processing circuitry 14 may be located within any combination of external device system 12, medical device system 16, and another device or group of devices that are not illustrated in FIG. 1. As such, techniques and capabilities attributed herein to processing circuitry 14 may be attributed to any combination of external device system 12, medical device system 16, and other devices that are not illustrated in FIG. 1.

Medical device system 16 may include one or more devices that may be attached to the body of patient 4. In some examples, medical device system 16 may include a wrist device (e.g., a wrist band including one or more sensors) for attaching to a wrist of the patient 4. In some examples, the wrist device may include a smart watch. In some examples, medical device system 16 may include a finger clip or ring device or another kind of finger device for attaching to a finger of patient 4. Medical device system 16 may include one or more optical sensors configured to generate optical signals indicative of one or more physiological parameters. In some examples, the one or more physiological parameters may include heart rate, respiration rate, pulse oximetry (SpO₂), regional oxygen saturation (rSO₂), pulse transit time (PTT), a photoplethysmography (PPG) value, blood pressure, hemoglobin content value, or any combination thereof.

A PPG signal may be indicative of a perfusion of blood to the dermis and subcutaneous tissue of patient 4. In this way, the PPG signal may represent a pulse of patient 4, where the PPG signal rises during a pulse and falls during periods between pulses. The PPG signal may reflect each heartbeat of patient 4, with a PPG peak corresponding to a heartbeat. In some examples, wearable device is located at an extremity (e.g., a finger, a wrist, a toe, or an ankle) of patient 4.

Processing circuitry 14 may be configured to measure one or more PTT intervals. A PTT interval may represent an amount of time between a ventricular depolarization (e.g., R-wave or other EGM feature indicative of depolarization) of a heart of patient 4 and a subsequent peak or other feature of the PPG signal corresponding to the contraction resulting from the ventricular depolarization. Each peak of the PPG signal may represent a maximum blood perfusion level in tissue proximate to medical device system 16 during a respective heart cycle. During a heart cycle, the ventricles contract, causing blood to flow from the heart through the vasculature before returning to the heart via the atria. As such, a PTT interval may represent an amount of time that it takes for an example blood cell to flow from the ventricle of patient 4 to the tissue proximate to an optical sensor of medical device system 16.

Although some example PTT intervals are described herein as being an amount of time between a ventricular depolarization of a heart of patient 4 and a subsequent peak of the PPG signal, processing circuitry 14 may define a PTT or another interval as having one or more different starting points and one or more different ending points. In some examples, the one or more starting points and the one or more ending points may include any combination of an atrial depolarization (e.g., a P-wave of the EGM signal), a repolarization of the ventricles (e.g., a T-wave of the EGM signal), a PPG signal valley, and a PPG signal inflection point). For example, processing circuitry 14 may measure a PTT interval as being an interval between an atrial depolarization and a PPG valley of the PPG signal, where the PPG valley corresponds to the same heartbeat (e.g., heart cycle) as the P-wave. In some examples, processing circuitry 14 may measure a PTT interval as being an interval between two fiducial points on the PPG signal. For example, the processing circuitry 14 may calculate a derivative of the PPG signal. The processing circuitry 14 may determine a PTT interval as being an amount of time between a peak of the derivative of the PPG signal and a peak of the PPG signal. Processing circuitry 14 may calculate the PPT interval as being an amount of time between any two fiducial points in a heart cycle.

One way that processing circuitry 14 may detect peaks in a signal is for processing circuitry 14 to calculate a derivative (e.g., difference) of the respective signal and identify one or more “zero crossings” of the signal. For example, to calculate one or more PPG peaks in the PPG signal, processing circuitry 14 may calculate a derivative of the PPG signal. Subsequently, in some cases, processing circuitry 14 is configured to identify a set of positive-going-negative zero crossings and a set of negative-going-positive zero crossings in the derivative of the PPG signals. The set of positive-going-negative zero crossings may represent relative peaks of the PPG signal, since a positive-going-negative zero crossing in the derivative of the PPG signal represents a point in which a slope of the PPG signal changes from being a positive slope to being a negative slope. The set of negative-going-positive zero crossings may represent relative valleys of the PPG signal, since a negative-going-positive zero crossing in the derivative of the PPG signal represents a point in which a slope of the PPG signal changes from being a negative slope to being a positive slope.

In some examples, to identify the one or more PPG peaks in the PPG signal, processing circuitry 14 may implement a “blanking window” following each detected PPG signal of the one or more PPG signals. For example, processing circuitry 14 may start a blanking window following a detected PPG peak in order to cause processing circuitry 14 to disregard any positive-going-negative zero crossings in the PPG signal which occur during the blanking window which extends for a period of time after the detected PPG peak. In some examples, processing circuitry 14 sets the length of the blanking window based on a heart rate of patient 4. For example, processing circuitry 14 may set the blanking window to a first length if patient 4 has a first hear rate and processing circuitry 14 may set the blanking window to a second length if patient 4 has a second heart rate, where the first blanking window is longer than the second blanking window if the first heart rate is lower than the second heart rate, and where the first blanking window is shorter than the second blanking window if the first heart rate is higher than the second heart rate. In some examples, processing circuitry 14 only detects one PPG peak per heart cycle, and the blanking window may prevent processing circuitry 14 from detecting more than one PPG peak per heart cycle.

Medical device system 16 may include one or more pressure devices configured to apply pressure to the patient 4. In some examples, medical device system 16 includes an air-filled cuff that can inflate to certain pressures based on a control circuit and air pump. In some examples, the cuff is located on a finger of patient 4. In some examples, the cuff is located on an arm of patient 4 (e.g., on a bicep). In any case, the cuff may be located “upstream” from one or more optical sensors of the medical device system 16 such that when the cuff inflates, the cuff induces hypoxia at an area proximate to where the one or more optical sensors are located. In some examples, the medical device system 16 includes a mechanical tourniquet to stop blood flow. This tourniquet may include visual cues on a tensioning device that allows for optimal tensioning of the tourniquet. In some examples, the tourniquet may include a pressure sensitive film that changes color when the correct pressure is reached. Additionally, or alternatively, the medical device system 16 may include a variable tourniquet that is a part of a feedback loop. Processing circuitry 14 may control a length of a band of the variable tourniquet, where the band wraps around a portion of the patient’s body. In some examples, medical device system 16 may apply pressure at a finger of the patient 4 mechanically using a spring, a stepper motor, a direct current (DC) motor, a servo, or any combination thereof.

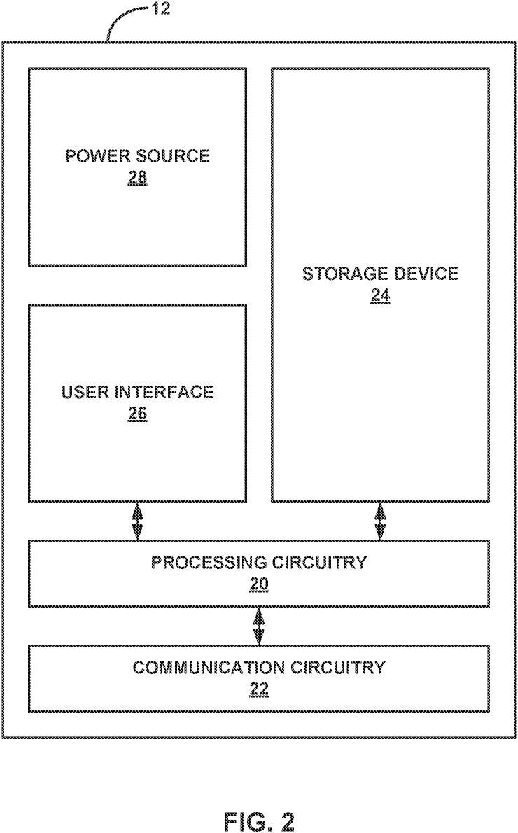

FIG. 2 is a block diagram illustrating an example configuration of components of external device system 12, in accordance with one or more techniques of this disclosure. In the example of FIG. 2, external device system 12 includes processing circuitry 20, communication circuitry 22, storage device 24, user interface 26, and power source 28.

Processing circuitry 20, in one example, may include one or more processors that are configured to implement functionality and/or process instructions for execution within external device system 12. For example, processing circuitry 20 may be capable of processing instructions stored in storage device 24. Processing circuitry 20 may include, for example, microprocessors, DSPs, ASICs, FPGAs, or equivalent discrete or integrated logic circuitry, or a combination of any of the foregoing devices or circuitry. Accordingly, processing circuitry 20 may include any suitable structure, whether in hardware, software, firmware, or any combination thereof, to perform the functions ascribed herein to processing circuitry 20. Processing circuitry 20 may, in some examples, include a portion of processing circuitry 14 or all of processing circuitry 14.

Communication circuitry 22 may include any suitable hardware, firmware, software or any combination thereof for communicating with another device or system, such as medical device system 16. Under the control of processing circuitry 20, communication circuitry 22 may receive downlink telemetry from, as well as send uplink telemetry to medical device system 16, or one or more other devices or systems.

Storage device 24 may be configured to store information within external device system 12 during operation. Storage device 24 may include a computer-readable storage medium or computer-readable storage device. In some examples, storage device 24 includes one or more of a short-term memory or a long-term memory. Storage device 24 may include, for example, random access memories (RAM), dynamic random access memories (DRAM), static random access memories (SRAM), magnetic discs, optical discs, flash memories, or forms of electrically programmable memories (EPROM) or electrically erasable programmable read-only memories (EEPROM). In some examples, storage device 24 is used to store data indicative of instructions for execution by processing circuitry 20. Storage device 24 may be used by software or applications running on external device system 12 to temporarily store information during program execution.

Data exchanged between external device system 12 and medical device system 16 via communication circuitry 22 may include operational parameters. External device system 12 may transmit data including computer readable instructions which, when implemented by medical device system 16, may control medical device system 16 to change one or more operational parameters and/or export collected data. For example, processing circuitry 20 may transmit an instruction to medical device system 16 which requests medical device system 16 to export collected data (e.g., electrical representations of optical signal data and/or other kinds of sensor data) to external device system 12. In turn, external device system 12 may receive the collected data from medical device system 12 and store the collected data in storage device 24. Additionally, or alternatively, processing circuitry 20 may export instructions to medical device system 16 requesting medical device system 16 to update one or more operational parameters such as parameters for controlling how medical device system 16 collects data. For example, processing circuitry 20 may export instructions for one or more times at which to collect data, one or more instructions for operating sensor(s) of medical device system 16, one or more instructions for operating a pressure device of medical device system 16.

A user, such as a clinician or patient 4, may interact with external device system 12 through user interface 26. User interface 26 includes a display (not shown), such as an LCD or LED display or other type of screen, with which processing circuitry 20 may present information related to medical device system 16 (e.g., electrical representations of one or more optical signals obtained from an optical sensor, one or more physiological parameters resulting from analysis of electrical representations of one or more optical signals, and information corresponding to one or more patient conditions resulting from analysis of electrical representations of one or more optical signals). In addition, user interface 26 may include an input mechanism to receive input from the user. The input mechanisms may include, for example, any one or more of buttons, a keypad (e.g., an alphanumeric keypad), a peripheral pointing device, a touch screen, or another input mechanism that allows the user to navigate through user interfaces presented by processing circuitry 20 of external device system 12 and provide input. In other examples, user interface 26 also includes audio circuitry for providing audible notifications, instructions or other sounds to patient 4, receiving voice commands from patient 4, or both. Storage device 24 may include instructions for operating user interface 26 and for managing power source 28.

Power source 28 is configured to deliver operating power to the components of external device system 12. In some examples, power source 28 may include a battery and a power generation circuit to produce the operating power. In some examples, the battery is rechargeable to allow extended operation. Recharging may be accomplished by electrically coupling power source 28 to a cradle or plug that is connected to an alternating current (AC) outlet. In addition, recharging may be accomplished through proximal inductive interaction between an external charger and an inductive charging coil within external device system 12. In other examples, traditional batteries (e.g., nickel cadmium or lithium ion batteries) may be used. In other examples, power source 28 may represent a power source other than a battery.

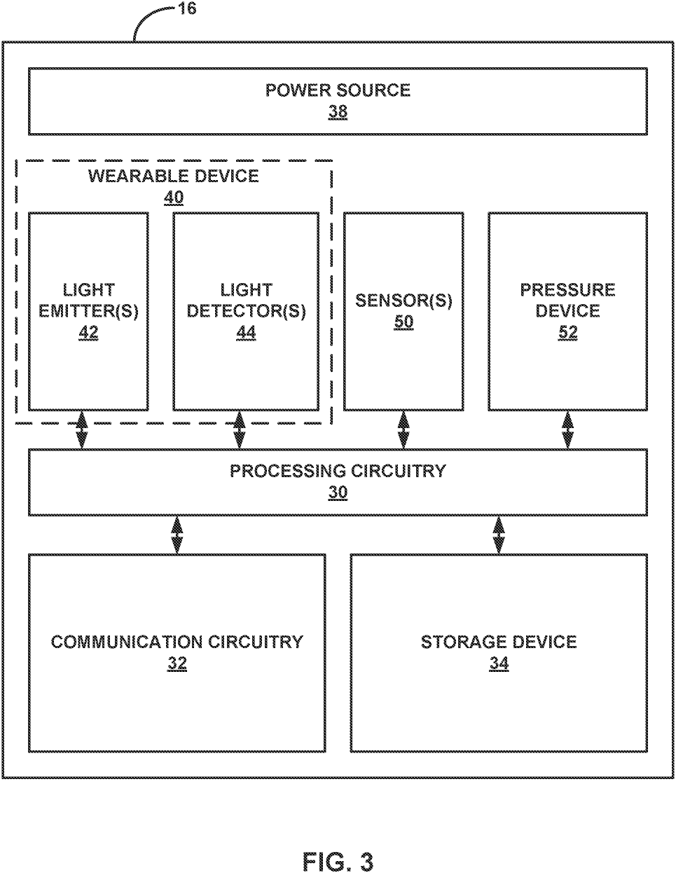

FIG. 3 is a block diagram illustrating an example configuration of components of medical device system 16, in accordance with one or more techniques of this disclosure. In the example of FIG. 3, medical device system 16 includes processing circuitry 30, communication circuitry 32, storage device 34, power source 38, wearable device 40, sensor(s) 50, and pressure device 52. Wearable device 40, which may also be referred to herein as a wearable sensor, may include light emitter(s) 42 and light detector(s) 44.

Processing circuitry 30, in one example, may include one or more processors that are configured to implement functionality and/or process instructions for execution within medical device system 16. For example, processing circuitry 30 may be capable of processing instructions stored in storage device 34. Processing circuitry 30 may include, for example, microprocessors, DSPs, ASICs, FPGAs, or equivalent discrete or integrated logic circuitry, or a combination of any of the foregoing devices or circuitry. Accordingly, processing circuitry 30 may include any suitable structure, whether in hardware, software, firmware, or any combination thereof, to perform the functions ascribed herein to processing circuitry 30. Processing circuitry 30 may, in some examples, include a portion of processing circuitry 14 of FIG. 1 or all of processing circuitry 14. Processing circuitry 14 and/or processing circuitry 30 (collectively, “processing circuitry 14, 30”) may perform any of the one or more techniques described herein.

Communication circuitry 32 may include any suitable hardware, firmware, software or any combination thereof for communicating with another device, such as external device system 12. Under the control of processing circuitry 30, communication circuitry 32 may receive downlink telemetry from, as well as send uplink telemetry to external device system 12, or another device.

Storage device 34 may be configured to store information within medical device system 16 during operation. Storage device 34 may include a computer-readable storage medium or computer-readable storage device. In some examples, storage device 34 includes one or more of a short-term memory or a long-term memory. Storage device 34 may include, for example, RAM, DRAM, SRAM, magnetic discs, optical discs, flash memories, or forms of EPROM or EEPROM. In some examples, storage device 34 is used to store data indicative of instructions for execution by processing circuitry 30. Storage device 34 may be used by software or applications running on medical device system 16 to temporarily store information during program execution.

Power source 38 is configured to deliver operating power to the components of medical device system 16. Power source 38 may include a battery and a power generation circuit to produce the operating power. In some examples, the battery is rechargeable to allow extended operation. Recharging may be accomplished by electrically coupling power source 38 to a cradle or plug that is connected to an alternating current outlet. In addition, recharging may be accomplished through proximal inductive interaction between an external charger and an inductive charging coil within medical device system 16. In other examples, traditional batteries (e.g., nickel cadmium or lithium ion batteries) may be used. Additionally, or alternatively, medical device system 16 may be directly coupled to an alternating current outlet to operate.

Medical device system 16 may include a wearable device 40 comprising light emitter(s) 42 and light detector(s) 44 configured to perform one or more optical signal measurements (e.g., PPG measurements, SpO₂ measurements, rSO₂ measurements, and PTT measurements). Although FIG. 3 illustrates light emitter(s) 42 and light detector(s) 44, as being part of a wearable device 40, this is not required. Medical device system 16 may include one or more non-wearable devices that comprise light emitter(s) 42 and light detector(s) 44. Light emitter(s) 42 may include one or more light emitting elements (e.g., LEDs). To perform one or more optical signal measurements, wearable device 40 may emit one or more output optical signals via light emitter(s) 42 and receive one or more return optical signals via light detector(s) 44. In some examples, one or more parameters of the output optical signals and/or one or more parameters of the return optical signals may indicate one or more physiological parameters of the patient 4. For example, the one or more parameters of the output optical signals and/or one or more parameters of the return optical signals may include one or more wavelength parameters, one or more magnitude and/or amplitude parameters, one or more phase parameters, or any combination thereof. Wearable device 40 and/or one or more other devices may generate an electrical representation of each optical signal emitted and collected by light emitter(s) 42 and light detector(s) 44. Wearable device 40 may output electrical representations of one or more optical signals to processing circuitry (e.g., processing circuitry 14, 30).

To perform a PPG measurement, light emitter(s) 42 may illuminate tissue of patient 4 proximate to medical device system 16. Light detector(s) 44 may collect one or more optical signals which indicate an amount of light absorbed by the tissue proximate to medical device system 16. In other words, light detector(s) 44 may detect at least some photons emitted by light emitter(s) 42 and reflected by the tissue proximate to light emitter(s) 42. An amount of light from light emitter(s) 42 that is detected by light detector(s) 44 may, in some examples, be inversely proportional to an amount of light from light emitter(s) 42 that is absorbed by the tissue of patient 4, but this is not required. The amount of light absorbed by the tissue may be correlated with a volume of blood present in the tissue proximate to medical device system 16. In this way, the PPG signal may include information indicative of one or more heart cycles of patient 4.

For example, each heart cycle of patient 4 may include a period of time in which a volume of blood in peripheral vasculature of patient 4 is elevated as compared with the rest of the heart cycle. Such a period of time may represent a PPG peak. The PPG data collected by light detector(s) 44 may include a PPG peak corresponding to each heart cycle which occurs during a PPG measurement performed by medical device system 16. In some examples, processing circuitry 14, 30 may determine one or more PTT intervals based on PPG signals collected by medical device system 16. The one or more PTT intervals may indicate a blood pressure of patient 4 or a factor of the blood pressure of patient 4.

In some examples, processing circuitry 14, 30 may perform pulse wave analysis (PWA) in order to determine one or more physiological parameters of patient 4. Processing circuitry 14, 30 may perform PWA in order to determine a blood pressure, a cardiac output (CO), an arterial stiffness, or other physiological parameters of patient 4. In some examples, processing circuitry 14, 30 may perform PWA based on a measured PPG signal.

In some examples, to perform an SpO₂ measurement, an rSO₂ measurement, or any other measurement for determining oxygenation of blood cells, light emitter(s) 42 may emit light at one or more wavelengths within the visible (VIS) light spectrum and/or emit one or more wavelengths within the near-infrared (NIR) light spectrum. The combination of VIS and NIR wavelengths may help enable processing circuitry 14, 30 to distinguish oxygenated hemoglobin from deoxygenated hemoglobin in the tissue of patient 4, since as hemoglobin becomes less oxygenated, an attenuation of VIS light increases and an attenuation of NIR decreases. By comparing the amount of VIS light detected by light detector(s) 44 to the amount of NIR light detected by light detector(s) 44, processing circuitry 14, 30 may determine the relative amounts of oxygenated and deoxygenated hemoglobin in the tissue of patient 4. For example, if the amount of oxygenated hemoglobin in the tissue of patient 4 decreases, the amount of VIS light detected by light detector(s) 44 increases and the amount of NIR light detected by light detector(s) 44 decreases. Similarly, if the amount of oxygenated hemoglobin in the tissue of patient 4 increases, the amount of VIS light detected by light detector(s) 44 decreases and the amount of NIR light detected by light detector(s) 44 increases. Based on the relative amounts of oxygenated and deoxygenated hemoglobin, the processing circuitry may monitor one or more patient conditions (e.g., sickle cell anemia, aplastic anemia, iron deficiency anemia, thalassemia, vitamin deficiency anemia, or any combination thereof).

Sensor(s) 50 may include one or more physiological sensors, such as electrodes, optical sensors, chemical sensors, temperature sensors, acoustic sensors, motion sensors, or any combination thereof. Sensor(s) 50 may collect data that processing circuitry 14 may analyze in order to track one or more patient conditions in addition to or alternatively to the optical data collected by the light detector(s) 44 located on wearable device 40.

Pressure device 52 may include a device for applying pressure to patient 4. In some examples, pressure device 52 may include an air-filled cuff that can inflate to certain pressures. For example, processing circuitry 14, 30 may control an air pump to inflate the cuff so that the cuff applies a certain pressure to patent 4. In some examples, the cuff is located on a finger of patient 4. In some examples, the cuff is located on an arm of patient 4 (e.g., on a bicep). In any case, the cuff may be located “upstream” from one or more optical sensors (e.g., light emitter(s) 42 and light detector(s) 44) of the medical device system 16 such that when the cuff inflates, the cuff induces hypoxia at an area proximate to where the one or more optical sensors are located. In some examples, the pressure device 52 includes a mechanical tourniquet to stop blood flow. This tourniquet may include visual cues on a tensioning device that allows for optimal tensioning of the tourniquet. In some examples, the tourniquet may include a pressure sensitive film that changes color when the correct pressure is reached. Additionally, or alternatively, the pressure device 52 may include a variable tourniquet that is a part of a feedback loop. Processing circuitry 14 may control a length of a band of the variable tourniquet, where the band wraps around a portion of the patient’s body. In some examples, pressure device 52 may apply pressure at a finger of the patient 4 mechanically using a spring, a stepper motor, a direct current (DC) motor, a servo, or any combination thereof.

In some examples, processing circuitry 14, 30 may receive, from medical device system 16, an electrical representation of a first optical signal. In some examples, the electrical representation of the first optical signal indicates a first one or more optical signal parameters such as one or more wavelengths, one or more magnitudes and/or amplitudes, one or more phases, or any combination thereof. For example, the first one or more optical signal parameters may include a plurality of wavelength signals each including a wavelength value, one or more magnitudes and amplitudes, one or more phases, or any combination thereof. The first one or more optical signal parameters may include a set of output optical signal parameters and a set of return optical signal parameters. Based on the set of output optical signal parameters and a set of return optical signal parameters, processing circuitry 14, 30 may determine one or more physiological parameters of the patient 4. In some examples, processing circuitry 14, 30 may receive the electrical representation of the first optical signal in response to medical device system 16 collecting the first optical signal without applying pressure to the patient 4 using pressure device 52. In some examples, processing circuitry 14, 30 may output an instruction for medical device system 16 to perform an optical signal measurement in order to collect the first optical signal.

Processing circuitry 14, 30 may control pressure device 52 to apply pressure to the patient 4 in order to control one or more physiological parameters of patient 4 proximate to the wearable device 40 which includes light emitter(s) 42 and light detector(s) 44. For example, processing circuitry 14, 30 may control pressure device 52 to apply pressure to patient 4 in order induce hypoxia in an area of patient 4 where medical device system 16 performs one or more measurements. In some examples, processing circuitry 13, 30 may determine, based on one or more measured physiological parameters of patient 4, for example by sensor(s) 50, an amount of pressure to apply to patient 4 for a measurement. The one or more physiological parameters of patient 4 may include blood pressure. To induce hypoxia, it may be beneficial for pressure device 52 to apply a pressure to patient 4 that is greater than a systolic blood pressure of patient 4 so that pressure device 52 prevents blood from flowing to the area where medical device system 16 performs one or more measurements or decreases an amount of blood that flows to the area where medical device system 16 performs one or more measurements.

In some examples, processing circuitry 14, 30 may determine how much pressure to apply to the patient 4 based on a blood pressure of the patient determined based on one or more signals collected by medical device system 16. For example, medical device system 16 may collect one or more PPG signals and determine one or more PTT intervals based on the one or more PPG signals. In some examples, processing circuitry 14, 30 may determine a blood pressure of the patient 4 based on the one or more PTT intervals. In some examples, processing circuitry 14, 30 may receive information indicative of the blood pressure of patient 4 from one or more other devices (e.g., external device system 12).

In some examples, processing circuitry 14, 30 may receive, from medical device system 16, an electrical representation of a second optical signal. In some examples, the electrical representation of the second optical signal comprises a second one or more optical signal parameters such as one or more wavelengths, one or more magnitudes and/or amplitudes, one or more phases, or any combination thereof. For example, the second one or more optical signal parameters may include a plurality of wavelength signals each including a wavelength value, one or more magnitudes and amplitudes, one or more phases, or any combination thereof. The second one or more optical signal parameters may include a set of output optical signal parameters and a set of return optical signal parameters. Based on the set of output optical signal parameters and a set of return optical signal parameters, processing circuitry 14, 30 may determine one or more physiological parameters of the patient 4. In some examples, processing circuitry 14, 30 may receive the electrical representation of the second optical signal in response to medical device system 16 collecting the second optical signal while applying pressure to the patient 4 using pressure device 52. In some examples, processing circuitry 14, 30 may output an instruction for medical device system 16 to apply the pressure to patient 4 and to perform an optical signal measurement in order to collect the second optical signal.

Processing circuitry 14, 30 may determine, based on the electrical representation of the first optical signal and the electrical representation of the second optical signal, one or more patient conditions. For example, processing circuitry 14, 30 may determine the one or more patient conditions based on one or more parameters of the first optical signal which medical device system 16 collected without applying pressure to patient 4, and based on one or more parameters the second optical signal which medical device system 16 collected while applying pressure to patient 4. Processing circuitry 14, 30 may determine the one or more parameters of the first optical signal and the one or more parameters of the second optical signal by analyzing the electrical representation of the first optical signal and the electrical representation of the second optical signal, respectively.

One or more differences may exist between the first optical signal and the second optical signal, where at least some of the one or more differences are caused by pressure device 52 inducing hypoxia while collecting the second optical signal. For example, the first optical signal and the second optical signal may each include a set of wavelength signals corresponding to a frequency range. One or more wavelength signals of the set of wavelength signals at a lower end of the frequency range may indicate one or more patient conditions (E.g., anemia). These one or more wavelength signals of the set of wavelength signals at a lower end of the frequency range may be more useful for monitoring anemia when pressure device 52 induces hypoxia as compared with examples where hypoxia is not induced. But it still may be beneficial for medical device system 16 to collect one or more optical signals without applying pressure to the patient 4, so that processing circuitry 14, 30 may analyze differences between optical signals collected with pressure and optical sensors collected without pressure by analyzing electrical representations of these optical signals.

In some examples, after applying pressure to patient 4 using the pressure device 52, processing circuitry 14, 30 may control the pressure device 52 to release the pressure to the patient 4 over a first period of time. Processing circuitry 14, 30 may control the medical device system 16 to perform an optical signal measurement in order to collect a third optical signal. In some examples, the third optical signal may correspond to a second period of time that at least partially overlaps with the first period of time. This means that the third optical signal may indicate a return of one or more optical signals to a “baseline” state that exists when pressure device 52 does not apply pressure to patient 4. In some examples, one or more physiological parameters and/or a length of time corresponding to the return of the optical signals to baseline state may indicate a status of one or more patient conditions. For example, when the patient has anemia, a viscosity of the blood may increase as compared with patients that do not have anemia. Therefore, it may take longer for the one or more optical signals to return to baseline when the patient has anemia, and the amount of time that it takes for the one or more optical signals to return to baseline may indicate a presence and/or a severity of the one or more patient conditions. Processing circuitry 14, 30 may determine, based on any one or combination of the electrical representation of the first optical signal, the electrical representation of the second optical signal, and the electrical representation of the third optical signal, the one or more patient conditions.

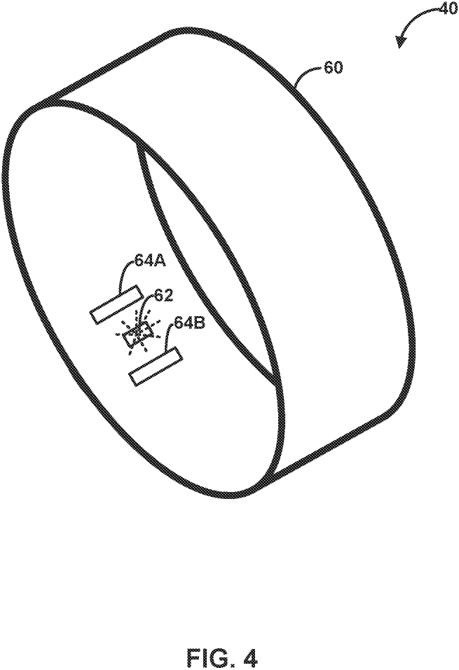

FIG. 4 is a conceptual drawing illustrating an example configuration of wearable device 40 of the medical device system 16 of FIG. 1 and FIG. 3, in accordance with one or more techniques of this disclosure. As seen in FIG. 4, wearable device 40 may include a band 60, but this is not required. Wearable device 40 may include any component that is configured to secure wearable device 40 to a body of patient 4. For example, wearable device 40 may include a clip, adhesive, another mechanical device, or any combination thereof in addition to or alternatively to the band 60. Light emitter 62 and light detectors 64A and 64B (hereinafter, “light detectors 64”) may be located on an interior surface of band 60 such that light emitter 62 and light detectors 64 face tissue of patient 4 when medical device system 16 is worn by patient 4. Light emitter 62 may be an example of light emitter(s) 42 of FIG. 3. Light detectors 64 may be an example of light detector(s) 44 of FIG. 3.

The example configuration of wearable device 40 illustrated in FIG. 4 may represent a ring for placement on a finger of patient 4. In other examples not illustrated in FIG. 4, wearable device 40 may include another device configured to be attached to the body of patient 4 such as a wrist bracelet, an ankle bracelet, a finger clip, or a smart device such as a smart watch. In any case, wearable device 40 may include light emitter 62 and light detectors 64 such that light emitter 62 may produce one or more optical signals and light detectors 64 may capture or receive one or more optical signals indicative of one or more physiological parameters.

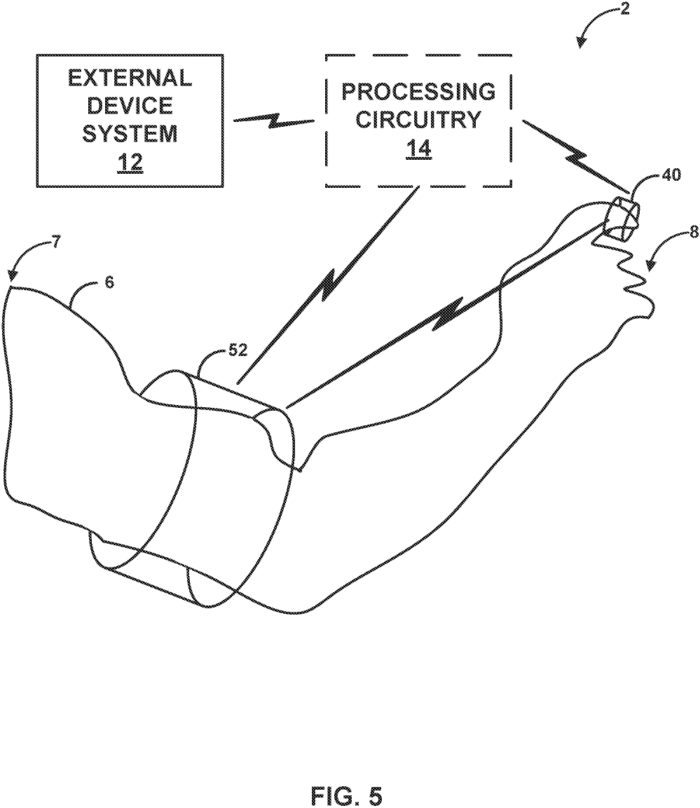

FIG. 5 is a conceptual drawing illustrating an example location of pressure device 52 and wearable device 40 on an arm 6 of patient 4, in accordance with one or more techniques of this disclosure. As seen in FIG. 5, wearable device 40 is located on a finger of the arm 6, and pressure device 52 is located on a bicep of the arm 6. This means that when processing circuitry 14 controls pressure device to apply pressure to the patient 4, the processing circuitry 14 may induce hypoxia in locations of the arm 6 that are “downstream” from the pressure device 52 relative to the patient’s bloodstream. The areas that are downstream from pressure device 52 include the finger on which wearable device 40 is located.

In some examples, processing circuitry 14 may be connected to wearable device 40 and/or pressure device 52 via wireless connections, wired connections, or any combination thereof. In some examples, at least a portion of processing circuitry 14 may be located as part of wearable device 40, pressure device 52, another device, or any combination thereof. In some examples, processing circuitry 14 may be completely separate from wearable device 40 and pressure device 52. In some examples, wearable device 40 may be connected to pressure device 52 via wireless connections, wired connections, or any combination thereof.

Processing circuitry 14 may be configured to control pressure device 52 to apply pressure to the patient 6. In some examples, processing circuitry 14 may be configured to control pressure device 52 to apply a specific amount of pressure for a specific amount of time. In some examples, processing circuitry 14 may control the specific amount of pressure and/or the specific amount of time based on information received from the external device 12. In some examples, processing circuitry 14 may control the specific amount of pressure and/or the specific amount of time based on information received from one or more sensors (e.g., one or more optical sensors located on wearable device 40). In some examples, rather than processing circuitry being configured to control pressure device 52, pressure device 52 may be controlled manually by a clinician.

Processing circuitry 14 may be configured to control wearable device 40 and/or pressure device 52 to perform one or more optical signal measurements. In some examples, processing circuitry 14 may be configured to control wearable device 40 to collect one or more optical signals without controlling pressure device 52 to apply pressure to the patient 4. In some examples, processing circuitry 14 may be configured to control wearable device 40 to collect one or more optical signals while controlling pressure device 52 to apply pressure to the patient 4. In some examples, processing circuitry 14 may be configured to control wearable device 40 to collect one or more optical signals while processing circuitry 14 controls pressure device 52 to release pressure from patient 4. In some examples, processing circuitry 14 may be configured to control wearable device 40 to collect one or more optical signals after processing circuitry 14 controls pressure device 52 to release pressure from patient 4.

The locations of wearable device 40 and pressure device 52 illustrated in FIG. 5 are not the only possible locations of wearable device 40 and pressure device 52. In some examples, wearable device 40 may need to be closer to a distal end 8 of the arm 6 than pressure device 52 in order to induce hypoxia in the location where wearable device 40 is located, but this does not mean that pressure device 52 needs to be located on a bicep and wearable device is located on a finger. In some examples, wearable device 40 may be located on a wrist of the arm 6, on the forearm of the arm 6, or on the upper arm of the arm 6. In some examples, the pressure device 52 may be located on the forearm of the arm 6, the wrist of the arm 6, or on a finger of the arm 6. In any case, the pressure device 52 may be located upstream from the wearable device 40 (e.g., the pressure device 52 is located closer to proximal end 7 of the arm 6 than wearable device 40).

Wearable device 40 and pressure device 52 are not limited to being located on an arm of patient 4. In some examples, Wearable device 40 and pressure device 52 may be located on one or more legs and/or one or more toes of patient 4. In some examples, the patient’s arm and fingers may be referred to herein as separate extremities. In some examples, the patient’s fingers may be referred to herein as being part of the same extremity as the rest of the patient’s arm.

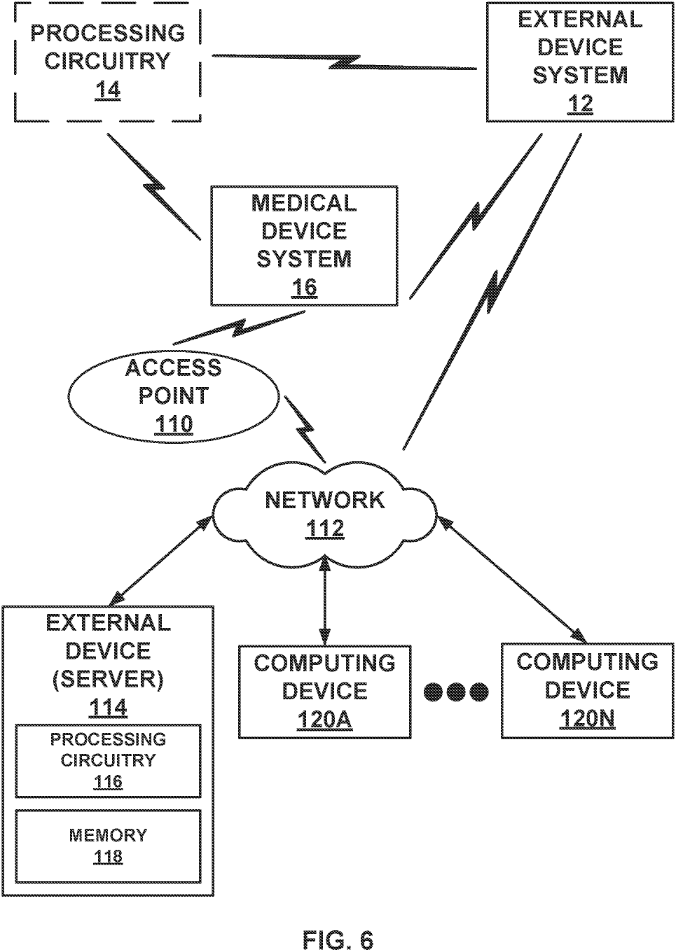

FIG. 6 is a block diagram illustrating an example system that includes an access point 110, a network 112, external computing devices, such as a server 114, and one or more other computing devices 120A-120N, which may be coupled to external device system 12, processing circuitry 14, and medical device system 16 via network 112, in accordance with one or more techniques of this disclosure. In this example, medical device system 16 may use communication circuitry to communicate with external device system 12 via a first wireless connection, to communicate with an access point 110 via a second wireless connection, and to communicate with medical device system 16 via a third wireless connection. In the example of FIG. 6, access point 110, external device system 12, medical device system 16, server 114, and computing devices 120A-120N are interconnected and may communicate with each other through network 112.

Access point 110 may include a device that connects to network 112 via any of a variety of connections, such as telephone dial-up, digital subscriber line (DSL), or cable modem connections. In other examples, access point 110 may be coupled to network 112 through different forms of connections, including wired or wireless connections. In some examples, access point 110 may be a user device, such as a tablet or smartphone, that may be co-located with the patient. As discussed above, medical device system 16 may be configured to transmit data, such as electrical representations of optical signal data. In addition, access point 110 may interrogate medical device system 16, such as periodically or in response to a command from the patient or network 112, in order to retrieve such signals or data, parameter values determined by medical device system 16, or other operational or patient data from medical device system 16. Access point 110 may then communicate the retrieved data to server 114 via network 112.

In some cases, server 114 may be configured to provide a secure storage site for data that has been collected from external device system 12 and/or medical device system 16. In some cases, server 114 may assemble data in web pages or other documents for viewing by trained professionals, such as clinicians, via computing devices 120A-120N.

Server 114 may include processing circuitry 116. Processing circuitry 116 may include fixed function circuitry and/or programmable processing circuitry. Processing circuitry 116 may include any one or more of a microprocessor, a controller, a DSP, an ASIC, an FPGA, or equivalent discrete or analog logic circuitry. In some examples, processing circuitry 116 may include multiple components, such as any combination of one or more microprocessors, one or more controllers, one or more DSPs, one or more ASICs, or one or more FPGAs, as well as other discrete or integrated logic circuitry. The functions attributed to processing circuitry 116 herein may be embodied as software, firmware, hardware or any combination thereof. In some examples, processing circuitry 116 may perform one or more techniques described herein based on electrical representations of optical signals, or data derived from these signals, or received from medical device system 16, as examples.

Server 114 may include memory 118. Memory 118 includes computer-readable instructions that, when executed by processing circuitry 116, cause processing circuitry 116 to perform various functions attributed to server 114 and processing circuitry 116 herein. Memory 118 may include any volatile, non-volatile, magnetic, optical, or electrical media, such as RAM, ROM, NVRAM, EEPROM, flash memory, or any other digital media.

In some examples, one or more of computing devices 120A-120N (e.g., device 120A) may be a tablet or other smart device located with a clinician, by which the clinician may program, receive alerts from, and/or interrogate medical device system 16. For example, the clinician may access data corresponding to an electrical representation of an optical signal collected by medical device system 16 through device 120A, such as when patient 4 is in between clinician visits, to check on a status of a medical. In some examples, the clinician may enter instructions for a medical intervention for patient 4 into an app in device 120A, such as based on a status of a patient condition determined by external device system 12, processing circuitry 14, medical device system 16, or any combination thereof, or based on other patient data known to the clinician. Device 120A then may transmit the instructions for medical intervention to another of computing devices 120A-120N (e.g., device 120B) located with patient 4 or a caregiver of patient 4. For example, such instructions for medical intervention may include an instruction to change a drug dosage, timing, or selection, to schedule a visit with the clinician, or to seek medical attention. In further examples, device 120B may generate an alert to patient 4 based on a status of a medical condition of patient 4, which may enable patient 4 proactively to seek medical attention prior to receiving instructions for a medical intervention. In this manner, patient 4 may be empowered to take action, as needed, to address his or her medical status, which may help improve clinical outcomes for patient 4.

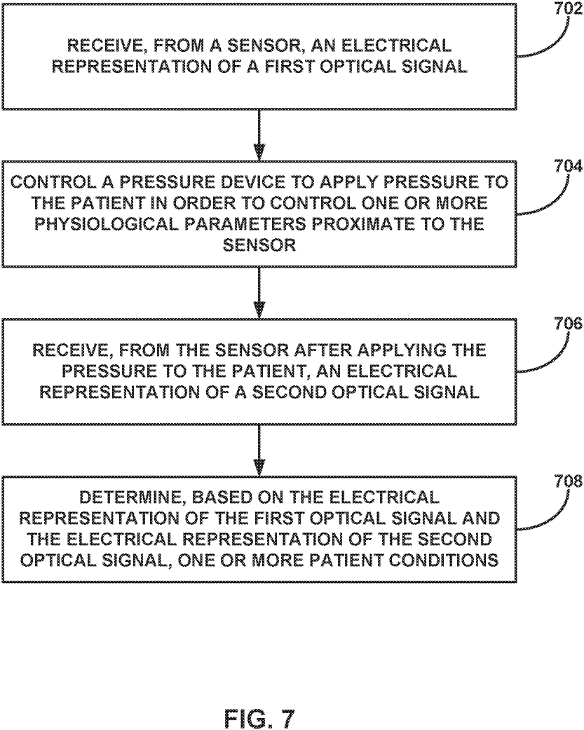

FIG. 7 is a flow diagram illustrating a first example operation for monitoring a patient condition, in accordance with one or more techniques of this disclosure. FIG. 7 is described with respect to external device system 12, processing circuitry 14, and medical device system 16 of FIGS. 1-6. However, the techniques of FIG. 7 may be performed by different components external device system 12, processing circuitry 14, and medical device system 16 or by additional or alternative medical device systems. Processing circuitry 14 is conceptually illustrated in FIG. 1 as separate from external device system 12, and medical device system 16 but may be any one or combination of processing circuitry of processing circuitry of external device system 12, and processing circuitry of medical device system 16. In general, the techniques of this disclosure may be performed by processing circuitry 14 of one or more devices of a system, such as one or more devices that include sensors that provide signals, or processing circuitry of one or more devices that do not include sensors, but nevertheless analyze signals using the techniques described herein. For example, another external device (not pictured in FIG. 1) may include at least a portion of processing circuitry 14, the other external device configured for remote communication with external device system 12, and/or medical device system 16 via a network.

In some examples, processing circuitry 14 may receive, from an optical sensor of medical device system 16, an electrical representation of a first optical signal (702). In some examples, the electrical representation of the first optical signal indicates one or more parameters. In some examples, processing circuitry 14 may control medical device system 16 to collect the electrical representation of the first optical signal when medical device system 16 is not applying pressure to patient 4. The one or more parameters may include a set of output signal parameters and a set of return signal parameters. Processing circuitry 14 may analyze the one or more parameters in order to monitor one or more patient conditions.

Processing circuitry 14 may control a pressure device 52 of medical device system 16 to apply pressure to the patient in order to control one or more physiological parameters proximate to the optical sensor of medical device system 16 (704). In some examples, processing circuitry 14 may control the pressure device 52 to apply an amount of pressure based on the blood pressure of the patient 4. Processing circuitry 14 may receive, from the sensor after applying the pressure to patient 4, an electrical representation of a second optical signal (706). In some examples, it may be beneficial for the optical sensor to collect one or more optical signals while applying pressure in order to induce hypoxia. Processing circuitry 14 may determine, based on the electrical representation of the first optical signal and the electrical representation of the second optical signal, one or more patient conditions (708).

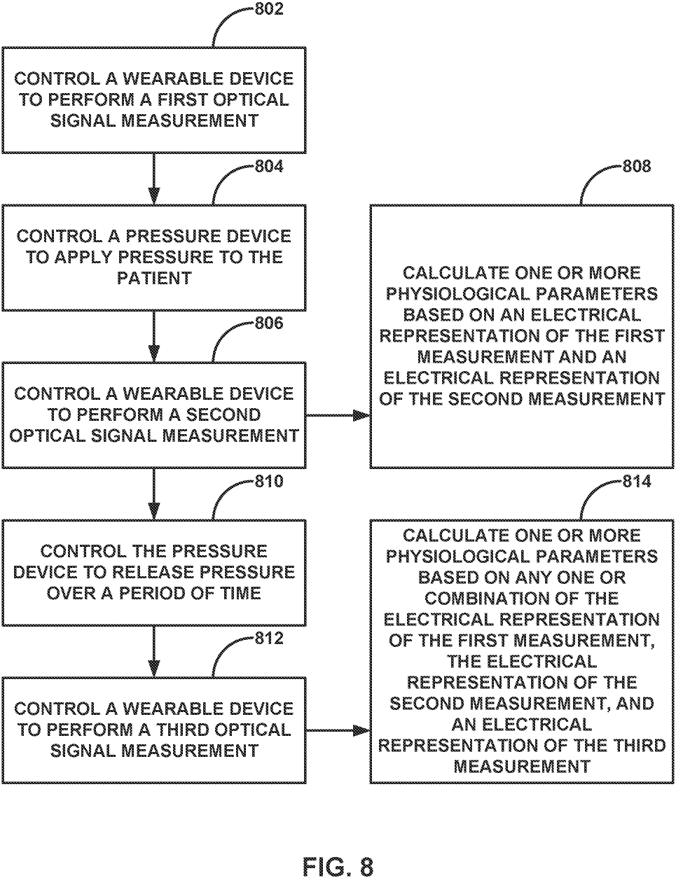

FIG. 8 is a flow diagram illustrating a second example operation for monitoring a patient condition, in accordance with one or more techniques of this disclosure. FIG. 8 is described with respect to external device system 12, processing circuitry 14, and medical device system 16 of FIGS. 1-6. However, the techniques of FIG. 8 may be performed by different components external device system 12, processing circuitry 14, and medical device system 16 or by additional or alternative medical device systems. Processing circuitry 14 is conceptually illustrated in FIG. 1 as separate from external device system 12, and medical device system 16 but may be any one or combination of processing circuitry of processing circuitry of external device system 12, and processing circuitry of medical device system 16. In general, the techniques of this disclosure may be performed by processing circuitry 14 of one or more devices of a system, such as one or more devices that include sensors that provide signals, or processing circuitry of one or more devices that do not include sensors, but nevertheless analyze signals using the techniques described herein. For example, another external device (not pictured in FIG. 1) may include at least a portion of processing circuitry 14, the other external device configured for remote communication with external device system 12, and/or medical device system 16 via a network.

Processing circuitry 14 may control a wearable device 40 to perform a first optical signal measurement (802). In some examples, to perform the first optical signal measurement, the wearable device 40 may emit one or more output optical signals and receive one or more return optical signals. In some examples, processing circuitry 14 may control the wearable device 40 to perform the first optical signal measurement when pressure device 52 is not applying pressure to the patient 4. In some examples, wearable device 40 may collect a first optical signal corresponding to the first optical signal measurement, and wearable device 40 may generate an electrical representation of the first optical signal for output to processing circuitry 14.

Processing circuitry 14 may control pressure device 52 to apply pressure to patient 4 (804). In some examples, by applying pressure to patient 4, pressure device 52 may induce hypoxia in an area of the patient where the wearable device 40 collects one or more optical signals. Processing circuitry 14 may control the wearable device 40 to perform a second optical signal measurement (806). In some examples, to perform the second optical signal measurement, the wearable device 40 may emit one or more output optical signals and receive one or more return optical signals. In some examples, processing circuitry 14 may control the wearable device 40 to perform the second optical signal measurement when pressure device 52 is applying pressure to the patient 4. In some examples, wearable device 40 may collect a second optical signal corresponding to the first optical signal measurement, and wearable device 40 may generate an electrical representation of the second optical signal for output to processing circuitry 14.

Processing circuitry 14 may calculate one or more physiological parameters based on an electrical representation of the first measurement and the electrical representation of the second measurement (808). The electrical representation of the first measurement may comprise an electrical representation of the first optical signal collected for the first measurement. The electrical representation of the second measurement may comprise an electrical representation of the second optical signal collected for the second measurement. In some examples, processing circuitry 14 may calculate heart rate, respiration rate, SpO₂, rSO₂, PTT, a PPG value, blood pressure, hemoglobin content value, or any combination thereof based on the electrical representation of the first measurement and the electrical representation of the second measurement.

Processing circuitry 14 may control pressure device 52 to release the pressure over a period of time (810). Processing circuitry 14 may control the wearable device 40 to perform a third optical signal measurement (812). In some examples, all or part of the third optical signal measurement may occur during the period of time when pressure device 52 releases the pressure. In some examples, all of the third optical signal measurement may occur after the period of time when the pressure device 52 releases the pressure. Processing circuitry 14 may calculate one or more physiological parameters based on any one or combination of the electrical representation of the first measurement, the electrical representation of the second measurement, and an electrical representation of the third measurement (814). In some examples, processing circuitry 14 may calculate heart rate, respiration rate, SpO₂, rSO₂, PTT, a PPG value, blood pressure, hemoglobin content value, or any combination thereof.

The following examples describe one or more techniques of this disclosure.

In some examples, a system includes a memory (e.g., any one or combination of storage device 24 and storage device 34) and processing circuitry (e.g., any one or combination of processing circuitry 14, processing circuitry 20, and processing circuitry 30) communicatively coupled to the memory. Processing circuitry 14, 20, 30 is configured to: receive, from a sensor (e.g., an optical sensor of wearable device 40), an electrical representation of a first optical signal; control pressure device 52 to apply pressure to patient 4 in order to affect one or more physiological parameters proximate to the optical sensor of wearable device 40; receive, from the optical sensor of wearable device 40 after applying the pressure to patient 4, an electrical representation of a second optical signal; and determine, based on the electrical representation of the first optical signal and the electrical representation of the second optical signal, one or more patient conditions.

In some examples, the electrical representation of the first optical signal indicates a first plurality of optical wavelength signals, the electrical representation of the second optical signal indicates a second plurality of optical wavelength signals, and processing circuitry 14, 20, 30 is configured to determine the one or more patient conditions based on the first plurality of optical wavelength signals and the second plurality of optical wavelength signals.

In some examples, wherein to determine the one or more patient conditions based on the first plurality of optical wavelength signals and the second plurality of optical wavelength signals, processing circuitry 14, 20, 30 is configured to: analyze the electrical representation of the first optical signal in order to determine a first set of optical wavelength signals of the first plurality of optical wavelength signals; determine, based on the first set of optical wavelength signals, one or more parameters corresponding to the electrical representation of the first optical signal; analyze the electrical representation of the second optical signal in order to determine a second set of optical wavelength signals of the second plurality of optical wavelength signals; and determine, based on the second set of optical signal wavelength signals, one or more parameters corresponding to the electrical representation of the second optical signal, wherein the first set of optical wavelength signals and the second set of optical wavelength signals correspond to a low end of an optical wavelength spectrum.

In some examples, the one or more parameters corresponding to the electrical representation of the first optical signal and the one or more parameters corresponding to the electrical representation of the second optical signal may include a heart rate, a respiration rate, a pulse oximetry (SpO₂) value, a regional oxygen saturation (rSO₂) value, a pulse transit time (PTT) value, a photoplethysmography (PPG) value, a hemoglobin content value, or any combination thereof. In some examples, to control the pressure device 52 to apply pressure to the patient, processing circuitry 14, 20, 30 is configured to: receive information indicative of a physiological parameter value of patient 4; determine, based on the physiological parameter value of patient 4, an amount of pressure to apply to patient 4; and control pressure device 52 to apply the amount of pressure to patient 4.

In some examples, the physiological parameter value of patient 4 includes a blood pressure of patient 4, and the processing circuitry 14, 20, 30 is configured to determine the amount of pressure to apply to patient 4 based on the blood pressure of patient 4.

In some examples, the optical sensor comprises a wearable optical sensor, the wearable optical sensor comprising light emitter(s) 42; and light detector(s) 44, and wherein the processing circuitry 14, 20, 30 is configured to control the wearable optical sensor to sense each optical signal of a set of optical signals by: controlling light emitter(s) 42 to emit an output optical signal comprising a plurality of output optical wavelength signals to the area of the patient’s body; and controlling light detector(s) 44 to receive a return optical signal comprising a plurality of return optical wavelength signals from the area of the patient’s body, wherein the return optical signal comprises the respective optical signal of the set of optical signals.

In some examples, the first optical signal corresponds to a first measurement, wherein the second optical signal corresponds to a second measurement, and wherein the processing circuitry 14, 20, 30 is further configured to: control the optical sensor of wearable device 40 to perform the first measurement while pressure device 52 is not applying pressure to patient 4; and control the optical sensor of wearable device 40 to perform the second measurement while the pressure device 52 is applying pressure to patient 4.

In some examples, pressure device 52 is placed at a first location on a first extremity of patient 4, the optical sensor of wearable device 40 is placed at a second location on a second extremity of patient 4, the second location is closer to a distal end of a limb (e.g., arm 6) of patient 4 than the first location, and wherein the processing circuitry 14, 20, 30 is configured to control the pressure device 52 to apply pressure to the first location, affecting the one or more physiological parameters proximate to the second location.

In some examples, the first extremity comprises arm 6 of the patient, wherein the first location comprises a bicep on the arm 6, wherein the second extremity comprises a finger at a distal end of arm 6, and wherein the second location comprises a location on the finger.

In some examples, by controlling the pressure device 52 to apply pressure to the first location on the extremity of patient 4, processing circuitry 14, 20, 30 is configured to induce hypoxia at the second location on the extremity of the patient during a time when the optical sensor of wearable device 40 senses the second optical signal.

In some examples, processing circuitry 14, 20, 30 is further configured to: control pressure device 52 to release the pressure to patient 4 over a first period of time; receive, from the optical sensor of wearable device 40 after releasing the pressure to patient 4, an electrical representation of the third optical signal; and determine, based on any one or combination of the electrical representation of the first optical signal, the electrical representation of the second optical signal, and the electrical representation of the third optical signal, the one or more patient conditions.

In some examples, wherein processing circuitry 14, 20, 30 is configured to: control pressure device 52 to release the pressure over a first period of time; and control the optical sensor of wearable device 40 to sense the third optical signal over a second period of time, wherein the second period of time at least partially overlaps with the first period of time.

In some examples, the one or more patient conditions comprise one or more blood conditions such as sickle cell anemia, aplastic anemia, iron deficiency anemia, thalassemia, vitamin deficiency anemia, or any combination thereof.

In some examples, a method includes receiving, by processing circuitry 14, 20, 30 communicatively coupled to a memory (e.g., any one or combination of storage device 24 and storage device 34), an electrical representation of a first optical signal; controlling, by processing circuitry 14, 20, 30, pressure device 52 to apply pressure to patient 4 in order to affect one or more physiological parameters proximate to the optical sensor of wearable device 40; receiving, by processing circuitry 14, 20, 30 from the optical sensor of wearable device 40 after applying the pressure to patient 4, an electrical representation of a second optical signal; and determining, by processing circuitry 14, 20, 30 based on the electrical representation of the first optical signal and the electrical representation of the second optical signal, one or more patient conditions.

In some examples, the electrical representation of the first optical signal indicates a first plurality of optical wavelength signals, the electrical representation of the second optical signal indicates a second plurality of optical wavelength signals, and the method further comprises determining, by processing circuitry 14, 20, 30, the one or more patient conditions based on the first plurality of optical wavelength signals and the second plurality of optical wavelength signals.

In some examples, determining the one or more patient conditions based on the first plurality of optical wavelength signals and the second plurality of optical wavelength signals comprises: analyzing, by processing circuitry 14, 20, 30, the electrical representation of the first optical signal in order to determine a first set of optical wavelength signals of the first plurality of optical wavelength signals; determining, by processing circuitry 14, 20, 30 based on the first set of optical wavelength signals, one or more parameters corresponding to the electrical representation of the first optical signal; analyzing, by processing circuitry 14, 20, 30, the electrical representation of the second optical signal in order to determine a second set of optical wavelength signals of the second plurality of optical wavelength signals; and determining, by the processing circuitry 14, 20, 30 based on the second set of optical signal wavelength signals, one or more parameters corresponding to the electrical representation of the second optical signal, wherein the first set of optical wavelength signals and the second set of optical wavelength signals correspond to a low end of an optical wavelength spectrum.

In some examples, controlling pressure device 52 to apply pressure to patient 4 comprises: receiving, by processing circuitry 14, 20, 30, information indicative of a physiological parameter value of patient 4; determining, by processing circuitry 14, 20, 30 based on the physiological parameter value of patient 4, an amount of pressure to apply to patient 4; and controlling, by processing circuitry 14, 20, 30, pressure device 52 to apply the amount of pressure to patient 4.

In some examples, the physiological parameter value of patient 4 includes a blood pressure of patient 4, and wherein the method further comprises determining, by processing circuitry 14, 20, 30, the amount of pressure to apply to patient 4 based on the blood pressure of patient 4.

In some examples, a non-transitory computer-readable medium comprising instructions for causing one or more processors (e.g., processing circuitry 14, 20, 30) to: receive an electrical representation of a first optical signal; control pressure device 52 to apply pressure to patient 4 in order to affect one or more physiological parameters proximate to the optical sensor of wearable device 40; receive, from the optical sensor of wearable device 40 after applying the pressure to patient 4, an electrical representation of a second optical signal; and determine, based on the electrical representation of the first optical signal and the electrical representation of the second optical signal, one or more patient conditions.

The techniques described in this disclosure may be implemented, at least in part, in hardware, software, firmware, or any combination thereof. For example, various aspects of the techniques may be implemented within one or more microprocessors, DSPs, ASICs, FPGAs, or any other equivalent integrated or discrete logic QRS circuitry, as well as any combinations of such components, embodied in external devices, such as physician or patient programmers, stimulators, or other devices. The terms “processor” and “processing circuitry” may generally refer to any of the foregoing logic circuitry, alone or in combination with other logic circuitry, or any other equivalent circuitry, and alone or in combination with other digital or analog circuitry.

For aspects implemented in software, at least some of the functionality ascribed to the systems and devices described in this disclosure may be embodied as instructions on a computer-readable storage medium such as RAM, DRAM, SRAM, magnetic discs, optical discs, flash memories, or forms of EPROM or EEPROM. The instructions may be executed to support one or more aspects of the functionality described in this disclosure.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements. The techniques of this disclosure may be implemented in a wide variety of devices or apparatuses, an external programmer, an integrated circuit (IC) or a set of ICs, and/or discrete electrical circuitry.

What is claimed is:

1. A system comprising:
a memory; and
processing circuitry communicatively coupled to the memory, wherein the processing circuitry is configured to:
receive, from a sensor, an electrical representation of a first optical signal;
control a pressure device to apply pressure to the patient in order to affect one or more physiological parameters proximate to the sensor;
receive, from the sensor after applying the pressure to the patient, an electrical representation of a second optical signal; and
determine, based on the electrical representation of the first optical signal and the electrical representation of the second optical signal, one or more patient conditions.

2. The system of claim 1, wherein the electrical representation of the first optical signal indicates a first plurality of optical wavelength signals, wherein the electrical representation of the second optical signal indicates a second plurality of optical wavelength signals, and wherein the processing circuitry is configured to determine the one or more patient conditions based on the first plurality of optical wavelength signals and the second plurality of optical wavelength signals.

3. The system of claim 2, wherein to determine the one or more patient conditions based on the first plurality of optical wavelength signals and the second plurality of optical wavelength signals, the processing circuitry is configured to:
analyze the electrical representation of the first optical signal in order to determine a first set of optical wavelength signals of the first plurality of optical wavelength signals;
determine, based on the first set of optical wavelength signals, one or more parameters corresponding to the electrical representation of the first optical signal;
analyze the electrical representation of the second optical signal in order to determine a second set of optical wavelength signals of the second plurality of optical wavelength signals; and
determine, based on the second set of optical signal wavelength signals, one or more parameters corresponding to the electrical representation of the second optical signal,
wherein the first set of optical wavelength signals and the second set of optical wavelength signals correspond to a low end of an optical wavelength spectrum.

4. The system of claim 3, wherein the one or more parameters corresponding to the electrical representation of the first optical signal and the one or more parameters corresponding to the electrical representation of the second optical signal may include a heart rate, a respiration rate, a pulse oximetry (SpO₂) value, a regional oxygen saturation (rSO₂) value, a pulse transit time (PTT) value, a photoplethysmography (PPG) value, a hemoglobin content value, or any combination thereof.

5. The system of claim 1, wherein to control the pressure device to apply pressure to the patient, the processing circuitry is configured to:
receive information indicative of a physiological parameter value of the patient;
determine, based on the physiological parameter value of the patient, an amount of pressure to apply to the patient; and
control the pressure device to apply the amount of pressure to the patient.

6. The system of claim 5, wherein the physiological parameter value of the patient includes a blood pressure of the patient, and wherein the processing circuitry is configured to determine the amount of pressure to apply to the patient based on the blood pressure of the patient.

7. The system of claim 1, wherein the sensor comprises a wearable optical sensor, the wearable optical sensor comprising:
one or more light emitters; and
one or more light detectors, and
wherein the processing circuitry is configured to control the wearable optical sensor to sense each optical signal of a set of optical signals by:
controlling the one or more light emitters to emit an output optical signal comprising a plurality of output optical wavelength signals to the area of the patient’s body; and
controlling the one or more light detectors to receive a return optical signal comprising a plurality of return optical wavelength signals from the area of the patient’s body, wherein the return optical signal comprises the respective optical signal of the set of optical signals.

8. The system of claim 1, wherein the first optical signal corresponds to a first measurement, wherein the second optical signal corresponds to a second measurement, and wherein the processing circuitry is further configured to:
control the sensor to perform the first measurement while the pressure device is not applying pressure to the patient; and
control the sensor to perform the second measurement while the pressure device is applying pressure to the patient.

9. The system of claim 1, wherein the pressure device is placed at a first location on a first extremity of the patient, wherein the sensor is placed at a second location on a second extremity of the patient, wherein the second location is closer to a distal end of a limb of the patient than the first location, and wherein the processing circuitry is configured to control the pressure device to apply pressure to the first location, affecting the one or more physiological parameters proximate to the second location.

10. The system of claim 9, wherein the first extremity comprises an arm of the patient, wherein the first location comprises a bicep on the arm, wherein the second extremity comprises a finger at a distal end of the arm, and wherein the second location comprises a location on the finger.

11. The system of claim 10, wherein by controlling the pressure device to apply pressure to the first location on the extremity of the patient, the processing circuitry is configured to induce hypoxia at the second location on the extremity of the patient during a time when the sensor senses the second optical signal.

12. The system of claim 1, wherein the processing circuitry is further configured to:
control the pressure device to release the pressure to the patient over a first period of time;
receive, from the sensor after releasing the pressure to the patient, an electrical representation of the third optical signal; and
determine, based on any one or combination of the electrical representation of the first optical signal, the electrical representation of the second optical signal, and the electrical representation of the third optical signal, the one or more patient conditions.

13. The system of claim 12, wherein the processing circuitry is configured to:
control the pressure device to release the pressure over a first period of time; and
control the sensor to sense the third optical signal over a second period of time, wherein the second period of time at least partially overlaps with the first period of time.

14. The system of claim 1, wherein the one or more patient conditions comprise one or more blood conditions such as sickle cell anemia, aplastic anemia, iron deficiency anemia, thalassemia, vitamin deficiency anemia, or any combination thereof.

15. A method comprising:
receiving, by processing circuitry communicatively coupled to a memory, an electrical representation of a first optical signal;
controlling, by the processing circuitry, a pressure device to apply pressure to the patient in order to affect one or more physiological parameters proximate to the sensor;
receiving, by the processing circuitry from the sensor after applying the pressure to the patient, an electrical representation of a second optical signal; and
determining, by the processing circuitry based on the electrical representation of the first optical signal and the electrical representation of the second optical signal, one or more patient conditions.

16. The method of claim 15, wherein the electrical representation of the first optical signal indicates a first plurality of optical wavelength signals, wherein the electrical representation of the second optical signal indicates a second plurality of optical wavelength signals, and wherein the method further comprises determining, by the processing circuitry, the one or more patient conditions based on the first plurality of optical wavelength signals and the second plurality of optical wavelength signals.

17. The method of claim 16, wherein determining the one or more patient conditions based on the first plurality of optical wavelength signals and the second plurality of optical wavelength signals comprises:
analyzing, by the processing circuitry, the electrical representation of the first optical signal in order to determine a first set of optical wavelength signals of the first plurality of optical wavelength signals;
determining, by the processing circuitry based on the first set of optical wavelength signals, one or more parameters corresponding to the electrical representation of the first optical signal;
analyzing, by the processing circuitry, the electrical representation of the second optical signal in order to determine a second set of optical wavelength signals of the second plurality of optical wavelength signals; and
determining, by the processing circuitry based on the second set of optical signal wavelength signals, one or more parameters corresponding to the electrical representation of the second optical signal,
wherein the first set of optical wavelength signals and the second set of optical wavelength signals correspond to a low end of an optical wavelength spectrum.

18. The method of claim 15, wherein controlling the pressure device to apply pressure to the patient comprises:
receiving, by the processing circuitry, information indicative of a physiological parameter value of the patient;
determining, by the processing circuitry based on the physiological parameter value of the patient, an amount of pressure to apply to the patient; and
controlling, by the processing circuitry, the pressure device to apply the amount of pressure to the patient.

19. The method of claim 18, wherein the physiological parameter value of the patient includes a blood pressure of the patient, and wherein the method further comprises determining, by the processing circuitry, the amount of pressure to apply to the patient based on the blood pressure of the patient.

20. A non-transitory computer-readable medium comprising instructions for causing one or more processors to:
receive an electrical representation of a first optical signal;
control a pressure device to apply pressure to the patient in order to affect one or more physiological parameters proximate to the sensor;
receive, from the sensor after applying the pressure to the patient, an electrical representation of a second optical signal; and
determine, based on the electrical representation of the first optical signal and the electrical representation of the second optical signal, one or more patient conditions.
